(19) United States
(12) Patent Application Publication
(10) Pub. No.: US 2004/0019197 A1
(43) Pub. Date: Jan. 29, 2004

(54) Method for producing liquid chromatography matrices

(76) Inventors: Nils Norrman, Uppsala (SE); Gunnar Glad, Uppsala (SE)

Correspondence Address: AMERSHAM BIOSCIENCES, PATENT DEPARTMENT, 800 CENTENNIAL AVENUE, PISCATAWAY, NJ, 08855, US

(21) Appl. No.: 10/451,193

(22) PCT Filed: Dec. 17, 2001
(86) PCT No.: PCT/EP01/14896

(30) Foreign Application Priority Data
Dec. 29, 2000 (SE) 0004929-6

(51) Int. Cl. C07H 5/04; C08B 37/00; C02F 1/28; B01D 15/08

(52) U.S. Cl. 536/55.3; 536/124; 210/656

(57) ABSTRACT

A method for the manufacture of a liquid chromatography matrix having affinity ligands, comprising the steps of: (i) providing a starting unfunctionalized liquid chromatography matrix (I) based on a polysaccharide; (ii) cross-linking the matrix by the use of a cross-linking agent in one or more cross-linking steps; and (iii) introducing the affinity ligands;Step (ii) is carried out with a cross-linking agent and to an extent requiring an increase of at least (10)% of acetonitrile in the eluant for eluting testosterone propionate from the matrix obtained in step (ii) compared to the percentage amount of the eluant required for eluting the same compound from the matrix provided in step (i). In a preferred variant the cross-linking (ii) is carried out to an extent increasing the maximal liquid flow velocity to ³(175)%, of the maximal flow velocity for matrix (I).

### TECHNICAL FIELD

The present invention concerns a new method for the manufacture of a functionalized chromatography matrix based on a polysaccharide. A matrix obtained by the novel method is able to withstand an increased liquid flow passing through the matrix in form of a packed bed or a monolith. Typical liquid flows are aqueous and above 5 cm/h.

This kind of matrices has found use in different kinds of liquid chromatography and corresponding batch-wise procedures, all of which primarily are based on affinity adsorption.

### BACKGROUND TECHNOLOGY

During more than thirty years, polysaccharide matrices have been commercially available for this kind of use. A typical manufacturing method has comprised the steps of:

(a) dissolving a polysaccharide in an aqueous liquid medium,

(b) transforming the polysaccharide to an insoluble form,

(c) optionally cross-linking the polysaccharide either simultaneously or subsequent to step (b), and

(d) functionalizing the polysaccharide.

This well-known production technology will give beads if the solution is emulsified in an organic solvent, which is not miscible with the aqueous liquid medium (water-in-oil emulsions). By including the proper cross-linking reagents, it will be possible to create inter- as well as intra-chain cross-links to an extent that will solidify the drops, i.e. an insoluble cross-linked 3-dimensional polymeric polysaccharide network will form. An alternative way to produce beads is to select a polysaccharide that dissolves in aqueous liquid media when warmed and solidifies to a gel when the temperature of the solution is decreased. In this latter variant the polysaccharide may be cross-linked simultaneously or subsequent to the gelling reaction.

In the case the solution is transformed to a gel without prior emulsification monolithic material will form.

Cross-linking is imperative for gel formation in case the polysaccharide is of the kind that lacks or has a too low gelling temperature. Otherwise cross-linking is optional and depends on use.

Cross-linking means that the rigidity of the material will increase which in turn means that the material may be better fitted to uses requiring application of pressure, such as in liquid chromatography.

The cross-linker can be introduced on the polysaccharide before or after the bead formation WO 9738018 (Amersham Pharmacia Biotech AB) and U.S. Pat. No. 4,975,683 (Amersham Pharmacia Biotech AB), respectively.

Polysaccharide material of this kind is always porous with pore sizes that primarily depend on the concentration of polysaccharide in the solution provided in step (a).

In the case monolithic material with very large pores are desired, fort instance ≧0.2 μm, it is preferred to form an oil-in-water solution that is transformed to a gel by cooling and/or cross-linking in the same manner as for a material with smaller pores. In the case beads with larger pores are desired, the oil-in-water emulsion is emulsified into an organic solvent that is immiscible with water. Finally the water phase is transformed to a gel in the same manner as discussed above. See U.S. Pat. No. 5,723,601 (Amersham Pharacia Biotech AB), WO 0017257 (Amersham Pharmacia Biotech AB) and WO 0012618 (Amersham Pharmacia Biotech AB).

Alternative ways of producing porous polysaccharide beads includes so-called atomisation techniques. These variants can be illustrated by spraying the solution in an air stream (WO 9702125 (FMC Corporation) and WO 0029466 (XC Corporation)) or by the so-called spinning disc atomisation (WO 9520620 (Biodev AB)).

Another alternative way is to coat individual solid particles with the polysaccharide solution prepared in step (a) and subsequently transform the solution to a gel (step (b)). The individual solid particles may be porous or non-porous. In the latter case internal as well as external surfaces of the particles may be coated.

### THE PROBLEMS SOLVED BY THE INVENTION

The cross-linking reaction increases the rigidity but in the typical case also the hydrophobicity meaning that certain drawbacks will appear. The rigidity determines the maximal liquid flow a chromatography matrix can withstand without collapsing. An increase in hydrophobicity means an increased risk for non-specific adsorption. The consequence of this has been that the maximal flow velocity has not set the limits but instead the balancing between a sufficient rigidity and an acceptable hydrophobicity. Many times, however, there has been a desire for matrices that permit higher maximal liquid flow velocities than what this principle has allowed.

The hydrophobicity of this kind of matrices can be measured by chromatographing a lipid-like neutral model molecule and comparing the retardation times or any other variable reflecting the strength between the model molecule and the matrix (Reubsaet et al., J. Chromatog. A 841 (1999) 147-154).

The rigidity of a matrix can be measured as the maximal flow velocity, the matrix can sustain in bed form before being fully compressed (before collapsing), i.e. to a stage where it does not permit any significant through-flow of liquid.

### OBJECTIVES OF THE INVENTION

The main objective of the invention is to provide a manufacturing method of the matrices mentioned above as well as the matrices as such that will permit an increased maximal liquid flow velocity while having an acceptable hydrophobicity.

### THE INVENTION

We have now recognised that this objective can be accomplished in case one starts from a polysaccharide matrix and cross-links it harder than usual, and then relies upon the functionalization step for obtaining the sufficient hydrophilicity.

The first aspect of the invention thus is a method for the manufacture of a liquid chromatography matrix having an affinity ligand, such as ion exchange groups. The final matrix may be in beaded or monolithic form. The method comprises the steps of:

(i) providing a starting unfunctionalized liquid chromatography matrix (I), which is based on a polysaccharide;

(ii) cross-linking the matrix by the use of a cross-linking reagent in one or more cross-linking steps; and

(iii) introducing the affinity ligand;

Step (iii) means that a plurality of the same or similar affinity ligands are introduced and results in matrix (II). The method is characterised in that step (ii) is carried out with a cross-linking reagent and to an extent requiring an increase of at least 10% of acetonitrile in the eluant for eluting testosterone propionate from the matrix obtained in step (ii) compared to the percentage amount of the eluant required for eluting the same compound from the matrix provided in step (i). In variants giving the most significant advantages, the increase is ≧25% such as ≧100% with the proviso that the eluant can never contain more than 100% acetonitrile. In addition to acetonitrile the eluant contain water. A typical absolute value for the starting unfunctionalized matrix is 1.5-5% acetonitrile and with the remaining part being water. The method used for measuring hydrophobicity is according to the method given in the experimental part.

By the term “unfunctionalized liquid chromatography matrix” is contemplated the affinity ligand is not present in the starting matrix (I).

In the preferred variants of the innovative method the cross-linking step (ii) is carried out to an extent increasing the maximal liquid flow velocity to ≧175%, such as to ≧250%, of the maximal flow velocity of the starting liquid chromatography matrix. The maximal liquid flow velocity is measured according to the method given in the experimental part.

The cross-linking reagent may be the same or different for the different cross-linking steps of step (ii).

If a certain way of introducing a particular type of cross-link requires more than one reagent all of them are included in the term “cross-linking reagent”.

There are mainly two kinds of cross-linking reagents that can be used:

(a) bifunctional reagents (including multifunctional reagents) in which each functional group is capable of reacting directly with the polysaccharide or an activated form thereof to give a covalent bond (homobifunctional reagents), and

(b) bifunctional reagents (including multifunctional reagents) in which there are at least two different functional groups that can be caused to react separately in time with the polysaccharide matrix (matrix (II)) (heterobifunctional reagents). Thus one functional group is typically reactive as such while another functional group of the reagent needs some kind of activation, for instance by being chemically transformed to a reactive group or by a change in the conditions provided by the reaction medium.

Directly reactive functional groups primarily are reactive with hydroxy groups and can be illustrated with electrophilic groups such as epoxides; haloalkyl groups such as halohydrins, vicinal dihalides, alpha-halocarbonyls etc; activated esters, acid halides etc.

Functional groups in the cross-linking reagent that require activation of the hydroxy group of the polysaccharide are typically nucleophilic, such as amino, hydroxy etc. Activation in this particular context typically means transformation to electrophilic groups, for instance of the type given in the preceding paragraph.

Bifunctional reagents of the second type (b) are illustrated by reagents in which the activatable function is an unsaturation, i.e. a carbon-carbon double or triple bond and the other function is represented by a group that is directly reactive with a hydroxy group in the matrix to be cross-linked or an activated form a hydroxy group. A directly reactive group of a cross-linking reagent can be selected according to the same principles as for type (a). Once inserted onto the matrix, halogenation and/or epoxidation may be used to activate the unsaturated group. Alternatively the group may be caused to react with each other, for instance via free radical reactions if they unsaturated. Typical examples of popular unsaturated groups are alkene groups such as in allyl and in acryl esters, acryl amides and the corresponding methacryl variants.

By the term “that a group is reactive with” means that it is able to react and form a covalent bond.

The cross-linking reagent may insert a cross-linking group that comprises a hydrocarbon group. Such a hydrocarbon group is bivalent, and may be linear, branched or cyclic and contain hydrogens and sp³-hybridised carbons. The cross-linking group may also comprise one or more of the groups: hydroxy, ether, thioether, keto, amido, ester etc, with the proviso that at most one atom selected from oxygen and sulphur binds to one and the same sp³-hybridised carbon.

The polysaccharide in the starting matrix (I) may be selected amongst dextran, agarose, cellulose, starch, pullulan etc, possibly derivatized to contain unchargeable hydrophilic groups that are pending to or cross-link the matrix. As a general rule this kind of hydrophilic groups typically has a ratio between oxygen atoms and carbon atoms that is ≧0.25 with due care taken that they are sufficiently stable against hydrolysis. This latter condition typically means that each sp³-hybridised carbon in the hydrocarbon group has at most one oxygen. The starting polysaccharide matrix may or may not be cross-linked.

The starting matrix as well as the matrix after step (iii) will always contain so-called micropores (smaller pores) in which mass transport is taking place by diffusion. In addition there may also be present macropores or superpores (larger pores) in which mass transport can take place by convection. The size range for the micropores typically extends up to 0.5 μm and is for the superpores 0.5-10 μm. For material in form of porous beads, the ratio between the pore diameters of the micropores may in the preferred variants extend up to 0.05 but is often below 0.01. The ratio between the pore diameters of the macropores and the bead diameter typically is in the interval 0.01-0.3, with preference for 0.05-0.2. See for instance WO 0017257 (Amersham Pharmacia Biotech AB), WO 0012618 (Amersham Pharmacia Biotech AB) and WO 9319115 (Amersham Pharmacia Biotech AB).

The matrix is preferably in beaded form but may also be in monolithic form, such as in form of a plug, a membrane, a filter etc.

For matrices in the form of beads, the mean bead diameter may vary depending on the use but as a general rule is within the interval of 1-1000 μm, preferably 1-50 μm for high performance applications and 50-300 μm for preparative purposes. A population of beads produced according to the invention may be mono disperse (mono sized) or poly dispersed (poly sized). By a mono disperse population of beads is contemplated that more than 95% of the beads have diameters (hydrodynamic diameters) within the mean diameter of the population ±5%.

Matrices in the form of beads may contain densifying particles resulting in a density above 1 g/cm³ for the beads if swollen in water. This kind of material is in particular adapted to be used in methods involving adsorptions to beads that have been fluidised by an upward liquid flow. See WO 9218237 (Amersham Pharmacia Biotech AB); WO 9717132 (Amersham Pharmacia Biotech AB); WO 9833572 (Amersham Pharmacia Biotech AB); and WO 9200799 (Kem-En-Tek/Upfront Chromatography A/S).

The beads may also be produced by so called atomisation techniques as discussed in general terms above.

Each bead of a given population of beads may contain one, two, three or more densifying particles per bead. Another variant is that all of the beads contain one single densifying particle.

In step (iii) the cross-linked matrix from step (ii) is functionalized with an affinity ligand enabling the use of the matrix in affinity adsorption and the like in order to bind a desired substance present in a liquid to the matrix.

The introduction of the affinity ligand may take place in one, two or more steps. In the normal cases one couples a compound that comprises the structure of the desired ligand to the matrix or a compound that gives the desired structure upon coupling. Typically the matrix is first activated before the ligand-forming compound is brought into the reaction mixture. The activation reagents may be either monofunctional or bifunctional, illustrative examples are cyanogen bromide, carbonyldiimazole, epichlorohydrine, allylglycidyl ether, reagents containing a thiol reacting group together with a hydroxy reacting group etc. Examples of thiol-reacting groups are reactive disulfides, alpha-halo carbopyls (esters, ketones etc), unsaturated groups conjugated to electron-withdrawing configurations etc. Examples of hydroxy reacting groups are activated esters etc. Altematively the ligand-forming compound may contain a functional group that is reactive with a hydroxy group.

Depending on the selected functionalisation chemistry, introduction of the affinity ligand may lead to a parallel cross-linking.

Typical affinity ligands are members of so called affinity pairs, more particularly bio-affinity pairs

The preferred affinity ligands are relatively small and/or have a pronounced hydrophilicity by having a large proportion of heteroatoms selected from oxygen, nitrogen and sulphur in relation to carbon. Typically the ligand-forming compounds have molecular weights that are at most 1000 dalton such as at most 700 dalton.

The preferred ligand-forming compounds introduce groups, which comprise a charged or chargeable moiety or group. Well-known such moieties are primary, secondary, tertiary and quatemary ammonium, amidinium, sulphonium, sulphonate, sulphate, phosphonate, phosphate, carboxy, phenolate etc.

Ligand-forming compounds introducing other kinds of affinity ligands may also be used provided the final ligand do not disturb the use of the matrix obtained after step (iii). Thus the final ligand should not disturb the hydrophilic/hydrophobic balance needed for a good compatibility with aqueous media and an acceptable level of unspecific adsorption. The ligand-forming compound thus may be selected as a member of well-known affinity pairs such as:

(a) antibodies and antigens/haptens,

(b) lectins and carbohydrate structures,

(c) IgG binding proteins and IgG (Protein A and IgG, Protein G and IgG etc),

(d) chelators and chelates,

(e) complementary nucleic acids,

(f) cells and cell binding ligands,

Potentially useful affinity members also include entities participating in catalytic reactions, for instance enzymes, enzyme substrates, cofactors, co-substrates etc. Members of cell-cell and cell-surface interactions and a synthetic mimetics of bio-produced affinity members are also included.

The invention will now be illustrated in the experimental part. The invention is further defined in the appending claims.

### EXPERIMENTAL PART

### Example 1

Determination of the Hydrophobicity of Separation Media.

This method is based on Reubsaet et al., J. Chromatog. A 841 (1999) 147-154. The selection of testosterone propionate, i.e. a neutral non-aromatic molecule, as the probe means a matching to the separation media tested.

Experimental:

Equipment: 2 Waters 510 HPLCV pumps, Waters 715 Ultra Wisp autoinjector, Waters 996 PDA detector, Waters System Interface Module, Millennium 2010 Data acq. Software and a LKB High Pressure Mixer.

ÄKTA™ purifier (APBiotech AB, Uppsala, Sweden), ÄKTA™ explorer 10XT (APBiotech AB), Shimadzu HPLC.

Columns: HR 5/% (APBiotech AB)

Chemicals: Water, testosterone propionate, acetonitrile, methanol.

Mobile phase A: MilliQ water

Mobile phase B: 95% (w/w) acetonitrile in MilliQ water (750 g acetonitrile+39.5 g water, total volume is 1001 ml).

Model substance (probe): 1 mM testosterone propionate (3.44 mg/10 ml) dissolved in methanol (the steroid dissolves faster when placed in an ultrasound bath).

| Method: | | | |
|---|---|---|---|
| Gradient elution: | 0%-100% B (0%-95% acetonitrile) in 70 min. | | |
| Flow: | 5 cm/min (1 ml/min on a HR5/5 column. | | |
| Injection volume: | 10 μl. | | |
| Injection: | 1^{st} blank injection with MeOH in vial position 1# | | |
| | 2^{nd} and 3^{rd} injection of testosterone propionate in vial | | |
| | position #2 | | |
| | Note place a vial with MeOH in position #3. | | |
| UV-detection: | 240 nm. | | |
| | Time | Mobile phase A | Mobile phase B |
| Start | 0 min | 100% | 0% |
| End gradient | 70 min | 0% | 100% |
| Rinse | 72 min | 0 | 100% |
| Start cond. | 73 min | 100% | 0% |
| End reequil. | 90 min | 100% | 0% |

Calculation of Result:

In order for the result to be system independent the retention times of the steroids must be normalised to their retention percentage of acetonitrile.

| | |
|---|---|
| Retention percentage acetonitrile = | %_{obs} |
| %_{obs} = | $( {t}_{r}^{obs} - Gradient\_delay ) \times [ \vert \frac{{\%}_{end} - {\%}_{start}}{Gradient\_length} \vert ] + {\%}_{start}$ |
| Gradient delay = | delay time from mixer to detector (column included). |
| | Gradient_delay must be determined in advance. |
| Gradient_length = | time gradient needed to reach maximum percentage |
| | acetonitrile: usually 70 min. |
| %_{end} = | end percentage of acetonitrile of the gradient, here 95% |
| %ₛₜₐᵣₜₜ = | start percentage of acetonitrile of the gradient, here 0 %. |

### Example 2

Testing for Maximal Liquid Flow Velocity.

Material:

HR 5/5 column with filters (APBiotech AB, Uppsala, Sweden).

At least 1 ml of chromatographic media in 20% EtOH or water.

A 10 ml Syringe with a 1/16 connection

20% EtOH or water to be used as packing eluant

Packing:

The bottom adaptor is mounted and the filter is wetted with 20% EtOH. The media slurry ca: 75% is added and the packing eluent is sucked through the column with the syringe. Further media is added until you have a packed bed height of 5 cm. A stop plug is mounted in the outlet of the column and the top adaptor is mounted and adjusted to the surface of the media.

Max Flow Test:

Testing Eluent: Water, 20% EtOH, 50% EtOH or whatever

Programmable pump: Akta system. See Example 1.

The packed columns are mounted in the pump system and the flow is increased with 0.5 ml each minute until the backpressure reaches 70 bar. The pressure/flow curve is printed and the max flow value is noted as the point where there is a sharp increase in the curve.

### Example 3

The Inventive Method.

Sepharose 6 Fast Flow (APBiotech AB, Uppsala, Sweden) is used as a starting model matrix. This matrix is based on agarose that has been cross-linked with epichlorohydrin The hydrophobicity measured as percentage acetonitrile at which testosterone propionate elutes is 2,5%. Its maximal liquid flow velocity is 7.5 cm/h.

A) Activation of Sepharose 6 FSDY FLOW with Allyl Glycidyl Ether.

A 100 g quantity (100 ml drained gel) of Sepharose 6 FAST FLOW was mixed with 15 ml of water, 45 ml of 50% aqueous solution of NaOH, 0.5 g of NaBH₄ and 13 g of Na₂SO₄. The mixture was stirred for 1 hour at 50° C. After addition of 100 ml of allylglycidyl ether the suspension was left at 50° C. under vigorous stirring for an additional 18 hours. After filtration of the mixture, the gel was washed successively, with 5×100 ml of distilled water, 5×100 ml of ethanol, 2×100 ml of distilled water, 2×100 ml of 0.2 M acetic acid, and 5×100 ml of distilled water. Titration gave a degree of substitution of 0.23 mmol of allyl/ml of gel.

The concentration of NaOH in the reaction described above is 5M. By increasing the NaOH concentration it is possible to increase the degree of substitution significantly. A 4 doubling of the NaOH concentration increased the degree of allyl group substitution from about 0.23 to about 0.7 mmol/ml of gel. The degree of substitution can also be varied by varying the amount of allyl glycidyl ether.

B) Activation of Allyl Sepharose 6 FAST FLOW Via Bromination.

Bromine was added to a stirred suspension of 100 ml of allyl activated Sepharose 6 FAST FLOW, 4 g of AcONa and 100 ml of distilled water, till a persistent yellow colour was obtained. Sodium formate was then added till the suspension was fully decolourised.

The reaction mixture was filtered and the gel washed with 5×100 ml of distilled water. The activated gel was then directly transfer to a reaction vessel and further reacted.

C) Cross-Linking.

A 100 g quantity (100ml drained gel) of bromine activated gel was mixed with 100 ml of water, 16 g of NaOH and 0.5 g of NaBH₄. The mixture was stirred For 16 hours at 50° C. After filtration of the mixture, the gel was washed successively, with 5×100 ml of distilled water, 2×100 ml of 0.2 M acetic acid and 5×100 ml of distilled water.

D) Q-Coupling & Cross-Linking.

100 g quantity (100 ml drained gel) of bromine activated gel was mixed with 25 ml of water and 50 ml of an aqueous solution of trimethylammonium chloride. After adjusting the pH to 11.5 with 50% aqueous solution of NaOH, the mixture was stirred for 16 hours at 25° C. After filtration of the mixture, the gel was washed sucessively, with 5×100 ml of distilled water, 2×100 ml of 0.5 M hydrochloric acid and 5×100 ml of distilled water.

The experiment above was repeated with variation in amount of allylglycidyl ether (cross-linker), base matrix (Sepharose 4 Fast Flow and Sepharose 6 Fast Flow) and with and without functionalization. The hydrophobicity and maximal flow velocity was determined according to examples 1 and 2, respectively. For the result see table 1.

TABLE 1
Hydrophobicity and maximal liquid flow velocity as function of
degree of allylation and introduction of an ion exchange ligand.

| Ex- | | | | | Hydro- | Maximal |
|---|---|---|---|---|---|---|
| peri- | Starting | Allylation | | | phobicity | flow |
| ment | matrix | μmol/ml gel | | Lig- | % | velocity |
| No | Sepharose | Step 1 | Step 2 | and | CH₃CN | cm/h |
| 1 | 6 Fast Flow | | | | 2.5 | 7.5 |
| 2 | 6 Fast Flow* | | | | | 12 |
| | (Q, S) | | | | | |
| 3 | 6 Fast Flow** | | | Q | 3.3 | 18 |
| 4 | 6 Fast Flow | 339 | | | 9.9 | 16 |
| 5 | 6 Fast Flow | 339 | | Q | 5 | 18.5 |
| 6 | 6 Fast Flow | 339 | 355 | Q | 3.7 | 33 |
| 7 | 6 Fast Flow | 339 | 634 | Q | 3.7 | 62 |
| 8 | 6 Fast Flow | 512 | | Q | 4.5 | 31 |
| 9 | 6 Fast Flow | 512 | | | 24 | 34 |
| 10 | 6 Fast Flow | 512 | 574 | 0 | 5,5 | 61 |
| 11 | 6 Fast Flow | 727 | | Q | 5.4 | 54 |
| 12 | 6 Fast Flow | 727 | | | 26.4 | 45 |
| 13 | 6 Fast Flow | 727 | 451 | 0 | 11.1 | 66 |
| 14 | 6 Fast Flow | 727 | 713 | Q | 4.2 | >75 |
| 15 | 4 Fast Flow | 212 | | | 6.2 | 14.5 |
| 16 | 4 Fast Flow | 212 | 460 | Q | 2.7 | 45 |
| 17 | 4 Fast Flow | 212 | 270 | Q | 2.6 | 22 |
| 18 | 4 Fast Flow | 212 | | Q | 2 | 11 |
| 19 | 4 Fast Flow | 600 | | | 23.7 | 38.5 |
| 20 | 4 Fast Flow | 600 | | Q | 6.5 | 42.5 |
| 22 | 4 Fast Flow | 600 | 370 | Q | 6.7 | 49 |
| 22 | 4 Fast Flow | 600 | 650 | Q | 3 | >75 |

Sepharose 6 Fast Flow and Sepharose 4 Fast Flow are based on a 6% and 4%, respectively, aqueous solution of agarose. Cross-linker epichlorohydrin. Both are commercially available from APBiotech AB, Uppsala, Sweden

* Unsubstituted base matrix for SP and Q Sepharose 6 Fast Flow. Not commercially available

** Q Sepharose 6 Fast Flow. Commercially available.

TABLE 2
Substitution degree, dry weight and maximal liquid flow velocity
after cross-linking

| | mmol | | |
|---|---|---|---|
| Sepharose 6 Fast Flow | allyl/ml gel | mg/ml gel | ml/min |
| Underivatised matrix | 0 | 58 | 7 |
| Allylated and cross-linked | 0.41 | 119 | 28 |
| Further allylated and cross-linked | 0.69 | 184 | 40 |

Sepharose 6 FAST FLOW is agarose beads that have been cross-linked with epichlorohydrin. It is apparent that allylation to 0.41 mmol of allyl will give a composite that comprise around 50% (w/w) of polysaccharide (agarose) and around 50% (w/w) of cross-linker.

Conclusion: A more than 100% increase in maximal liquid flow velocity can be accomplished for composite polysaccharide material in which the non-polysaccharide material constitutes of ≧40%, such that ≧50% or ≧60%, of the cross-linked material before an affinity ligand has been introduced. Similarly should apply after an affinity ligand has been introduced. The percentage is in w/w.

1. A method for the manufacture of a liquid chromatography matrix having affinity ligands, for instance charged ligands such as ion exchange groups, comprising the steps of:
i) providing a starting unfunctionalized liquid chromatography matrix (I) based on a polysaccharide;
ii) cross-linking the matrix by the use of a cross-linking agent in one or more cross-linking steps; and
iii) introducing the affinity ligands;
characterised in that step (ii) is carried out with a cross-linking agent and to an extent requiring an increase of at least 10% of acetonitrile in the eluant for eluting testosterone propionate from the matrix obtained in step (ii) compared to the percentage amount of the eluant required for eluting the same compound from the matrix provided in step (i), said measuring method being according to the method given in the experimental part.

2. The method of claim 1, characterised in that the matrix (I) and preferably also matrix (II) islare in the form of beads.

3. The method of any of claims 1-2, characterised in that the cross-linking (ii) is carried out to an extent increasing the maximal liquid flow velocity to ≧175%, such as to ≧250%, of the maximal flow velocity for matrix (I), the maximal liquid flow velocity being measured according to the method given in the experimental part.

4. The method of any of claims 1-3, characterised in that step (iii) is performed with reagent(s) that in a parallel reaction cause cross-linking.

5. The method of any of claims 1-4, characterised in that the cross-linking agent is the same or different in each cross-linking step of step (ii).

6. The method of any of claims 1-5, characterised in that the cross-linking agent has two or more groups each of which is capable of reacting with a hydroxy group or with an activated forms thereof, and that cross-linking groups created within the beads comprise hydrocarbon groups.

7. The method of claim 6, characterised in that the cross-linking agent is selected such that the cross-linking group comprises one or more groups selected from hydrocarbon groups that are linear, branched or cyclic and contain hydrogens and sp³-hybridised carbons, and hydroxy, ether, thioether, keto, amido, ester etc, with the proviso that at most one atom selected from oxygen and sulphur binds to one and the same sp³-hybridised carbon in the hydrocarbon group.

8. The method of any of claims 6-7, characterised in that the ratio between the number of carbon atoms and the sum of the number of oxygen and sulphur atoms is ≧3 in each hydrocarbon group.

9. The method of any of claims 6-8, characterised in that said two or more groups that are capable of reacting with a hydroxy group or an activated form thereof are selected amongst haloalkyl (X—CH₂— where X is a halogen atom) epoxy, activated ester etc.
